(19) United States
(12) Patent Application Publication
(10) Pub. No.: US 2019/0241537 A1
(43) Pub. Date: Aug. 8, 2019

(54) Odorless Cannabis Products and Processes for Their Preparation

(71) Applicant: Seth Dramen, San Francisco, CA (US)

(72) Inventor: Seth Dramen, San Francisco, CA (US)

(21) Appl. No.: 16/267,383

(22) Filed: Feb. 4, 2019

Related U.S. Application Data
(60) Provisional application No. 62/625,641, filed on Feb. 2, 2018.

(51) Int. Cl. C07D 311/80; B01D 3/38; B01D 3/40; C07B 63/00

(52) U.S. Cl. CPC C07D 311/80; C07B 63/00; B01D 3/40; B01D 3/38

(57) ABSTRACT

Cannabis plant products, and processes for their preparation using hydrodistillation and steam distillation, are described, where such products are measurably or detectably free from terpenes, and thereby odorless, but where they substantially contain the same cannabinoid profile and content, and have the same visual and tactile quality as the original cannabis plant material from which they are obtained.

CROSS-REFERENCE TO RELATED APPLICATIONS: The present application claims priority to and the benefit of U.S. Provisional Patent Application No. 62/625,641, filed on Feb. 2, 2018, which is incorporated herein by reference in its entirety for all purposes.

### FIELD OF THE INVENTION

The present disclosure generally relates to cannabis plant products, and processes for their preparation using water and steam distillation.

### BACKGROUND OF THE INVENTION

Cannabis flowers, and the smoke from cannabis flowers when consumed, have a distinct and unmistakable odor. That fragrance, immediately recognizable to people around the world, comes from a class of chemicals in the cannabis plant known as terpenes. Terpenes are volatile and highly potent, and while each individually adds a different aroma to the final mix—whether it be piney, citrusy, earthy, peppery, floral, herbal, or another—taken together the terpenes make the fragrance identifiable as just one thing: cannabis. Cannabis smoke or even cannabis flower alone therefore quickly signal to people that cannabis is nearby.

To some, the rich and pungent fragrance of cannabis is not only enjoyable but one of its best attributes. Further, the particular fragrance created by the specific combination of terpenes in a cannabis strain is useful both to help identify it, as well as to help determine the different effects that consumption of that strain might cause. Even so, the strong fragrance of cannabis and cannabis smoke is frequently undesirable. While cannabis may be consumed for medicinal and adult-use purposes throughout more and more of the country, cannabis currently remains on Schedule I of the Controlled Substances Act, and federal prohibition continues. Accordingly, even though public perception is shifting, still some may not want to create a fragrance that is a dead giveaway to consumption. Apart from just privacy and discretion, some also may want to be mindful of neighbors, or respectful of others in a public setting.

In view of these concerns, numerous methods have long been used to hide the fragrance of cannabis and cannabis smoke. Methods include masking the fragrance by burning something with another strong smell such as incense, sage, or scented candles; by using aromatherapy diffusers; and with chemical sprays and odor eliminators. Other methods include absorbing the fragrance, passing it through filters and air purifiers, and diffusing it with fans. Some, out of consideration for others, may avoid smoking in their homes or in public places altogether. But none of these solutions is ideal; and indeed, until the present invention, no solution has existed that would allow one to smoke cannabis flower without creating as a byproduct the fragrance that has been forever associated with its consumption.

Cannabis

Cannabis is a genus of flowering plant in the family Cannabaceae that is commonly recognized as containing the three species Cannabis sativa, Cannabis indica, and Cannabis ruderalis. Cannabis has a long history of use for industrial purposes (e.g., for fiber and oil), medicinal purposes (e.g., to treat the causes or improve the symptoms of disease), and recreational purposes (e.g., for its psychoactive effects). The two primary types of phytochemicals produced by cannabis plants are: First, the cannabinoids, which are in part responsible for the mental and physical effects of cannabis when consumed; and second, the terpenes, responsible for the unique fragrance of different cannabis strains, as well as for numerous mental and physical effects.

Cannabinoids

Cannabinoids are a diverse class of small molecules that are grouped together because of their ability to act on cannabinoid receptors found in the brain and throughout the central and peripheral nervous systems of humans and other mammals. There are two major types of cannabinoid receptors, known as cannabinoid 1 (CB₁) and cannabinoid 2 (CB₂). CB₁ receptors are found primarily in the central nervous system (i.e., the brain and spinal cord), as well as in the lungs, liver and kidneys. CB₂ receptors are found primarily in the immune system and in hematopoietic stem and progenitor cells (i.e., the cells in the bone marrow which differentiate into the various blood cell types).

Cannabinoids produced endogenously in humans and other mammals are termed endocannabinoids. Taken together, these endocannabinoids and the endogenous cannabinoid receptors they act on (along with the enzymes for their synthesis and degradation) form the endocannabinoid system. The endocannabinoid system has been shown or suggested to be involved in a variety of physiological processes including appetite, digestion, pain sensation, mood, memory, reproduction, stress response, immune function, thermoregulation, energy balance, and sleep.

Cannabinoids also have been isolated from plants, including at least 100 from the Cannabis plant, among other plants including echinacea, kava, tea, and flax. Cannabinoids from plants are termed phytocannabinoids. These non-endogenous cannabinoids also act on cannabinoid receptors in the body, and they have many structural and functional similarities with endogenous cannabinoids. Among the naturally-occurring phytocannabinoids from cannabis, tetrahydrocannabinol (THC), cannabidiol (CBD), and cannabinol (CBN) are the three major constituents.

Tetrahydrocannabinol is the most widely-known cannabinoid derived from the Cannabis plant, in large part because of its psychoactive effects. The psychoactive effects of THC are thought to be primarily because of its activation of CB_{'} receptors.

Cannabidiol, unlike THC, does not produce psychoactive effects in humans (and in fact, can antagonize those effects). However, CBD is reported to demonstrate numerous other pharmacological properties. For instance, CBD has been shown to exert analgesic, antioxidant, anti-inflammatory, antiemetic, anticonvulsant, antipsychotic, anxiolytic, antidepressant, anticompulsive, antitumoral, neuroprotective, and immunomodulatory effects. CBD exerts its effects primarily through its indirect interaction with both CB₂ and CB₁ receptors.

Cannabinol, the third major naturally-occurring phytocannabinoid found in cannabis, is weakly psychoactive, and found only in trace amounts. CBN has been shown to have analgesic properties but otherwise is thought to exert minimal pharmacological effects in the central nervous system. CBN acts upon CB₂ receptors, and also upon CB_{'} receptors (but with lower affinity than THC).

Other naturally-occurring phytocannabinoids found in cannabis include, among numerous more, cannabigerol (CBG), cannabinodiol (also known as cannabidinodiol) (CBDL, CBND), cannabichromene (CBC), cannabielsoin (CBE), cannabicyclol (CBL), cannabicitran (CBT), cannabivarin (CBV), cannabidivarin (CBDV), tetrahydrocannabivarin (THCV), cannabichromevarin (CBCV), cannabigerovarin (CBGV), and cannabigerol monomethyl ether (CBGM).

Notably, in fresh plant material the cannabinoids are almost exclusively found in their “acidic” form (as THCA and CBDA, for example), rather than their “neutral” form (e.g., as THC and CBD). Cannabinoids in their acidic form have an extra carboxyl group attached, which can be removed through a process called decarboxylation, wherein the carboxyl group is lost as carbon dioxide. The acidic forms of the cannabinoids may have some pharmacological properties, but generally must be converted into their neutral form before they are active and bioavailable in humans (for instance, it is THC, and not THCA, that is responsible for the psychoactive effects of consumed cannabis).

How quickly the process of decarboxylation occurs is a function of heat and time. At low temperatures, such as when a plant is dried and cured after harvesting, decarboxylation occurs very slowly and only partially. At high temperatures, such as when cannabis is combusted by smoking (i.e., at temperatures over 450° F.), the cannabinoids are almost instantaneously decarboxylated, and are thus immediately available for absorption through inhalation. At temperatures in between, such as when cannabis plant material is heated in boiling water (i.e., at 212° F. at atmospheric pressure), the cannabinoids may take 90 minutes to several hours or more to decarboxylate fully. See, e.g., Wang et al., Decarboxylation Study of Acidic Cannabinoids: A Novel Approach, Cannabis and Cannabinoid Res. 1.1:262-271 (2016).

Terpenes

Terpenes are a large and diverse class of organic compounds, produced by a broad variety of plants, including cannabis. The word “terpene” is derived from the word “turpentine,” the pungent resin extracted from the terebinth tree Pistacia terebinthus. The presence and particular combination of terpenes gives different plant varieties their distinctive smells and tastes. Due to their odiferous nature, terpenes are also common constituents of commercial flavorings and fragrances, are used as agents in food, candy, and perfume, and are sought after for aromatherapy. (Although often referred to as “aromatic” in the lay sense, because that word has a separate and specialized meaning in chemistry, “odiferous” is used herein to avoid confusion; note also that “odor” and “fragrance” are used interchangeably.)

In cannabis, terpenes form the largest group of phytochemicals, with at least 120 identified molecules. Terpenes generally make up between 10-20 percent of the total oil content produced by cannabis resin glands. Terpenes also constitute the majority of chemicals in the heated or vaporized smoke of cannabis flowers, often consisting of greater than 50%, with cannabinoids normally accounting for 10-20%.

Chemically, terpenes are constituted by one or more repeating units of the five-carbon building block known as an isoprene unit (having the molecular formula C₅H₈). These isoprene units may be linked together end-to-end to form linear chains, or may be arranged so as to form rings (thus having the molecular formula (C₅H₈)ₙ, where ₙ is the number of linked isoprene units). Terpenes are classified in families according to the number of isoprene units from which they are constituted: as hemiterpenes (one unit), monoterpenes (two), sesquiterpenes (three), diterpenes (four), sesterterpenes (five), triterpenes (six), sesquarterpenes (seven), tetraterpenes (eight), and polyterpenes (nine or more).

Monoterpenes generally dominate the terpene profile of cannabis. Monoterpenes include β-myrcene, d-limonene, α- and β-pinene, terpinolene and linalool. Sesquiterpenes (and β-caryophyllene and α-humulene in particular) also occur to a large extent in cannabis, as do triterpenes (as β-amyrin, friedelin and epifriedelanol, cycloartenol, and dammaradienol). Monoterpenes are more volatile and evaporate more easily than either sesquiterpenes or triterpenes.

Terpenes can also be referred to as “terpenoids” when they have experienced oxidation (for instance, after cannabis has been cured and dried), and have an additional oxygen-containing functional group. Typically however, the terms are used interchangeably, and the term “terpene” as used herein shall refer to both, unless stated otherwise.

The Role of Terpenes in Cannabis Odor

Different terpenes each have their own distinctive odor, which they may contribute to a broad variety of plants, including cannabis. For instance, β-myrcene, the most commonly found terpene in cannabis, and found often in the highest concentrations, is also present in mango, lemongrass, bay leaves, and hops. Likewise, β-caryophyllene is found not only in cannabis, but also in black pepper, cloves, rosemary, and hops. Linalool, found in cannabis, is responsible as well for the distinctive scent of lavender, and is therefore also used in numerous perfumed hygiene products and cleaning agents including soaps, detergents, shampoos, and lotions.

Some terpenes also exist in multiple chemical forms as isomers or enantiomers, each with a slightly different odor. For example, there are three closely related forms of limonene, which respectively smell predominantly of tangerine, lemon, and grapefruit. Combinations of these three forms of limonene can be used to create a spectrum of different aromas, depending on the amounts and ratios present.

Each cannabis strain (and often, even different phenotypes of the same strain) will have a particular mix of terpenes that are responsible for its characteristic odor and flavor. As such, these different terpene profiles have contributed to the selection of cannabis strains under human domestication. (“Strains” being used herein to refer to both “cultivars” and “chemovars,” the distinction between which is discussed, e.g., in Hazekamp and Fischedick, “Cannabis—From Cultivar to Chemovars,” Drug Testing and Analysis, 4(7-8):660 (2012) and Hazekamp et al, “Cannabis: From Cultivar to Chemovar II—A Metabolomics Approach to Cannabis Classification,” Cannabis and Cannabinoid Res. 1(1):202 (2016)). Indeed, patients and recreational consumers will often ask to smell a strain of cannabis when selecting it, and many believe that certain aromas, by demonstrating which terpenes are present in a strain, can identify particular effects that strain may produce upon consumption. Terpene “flavor wheels” have been developed to help consumers decide on their strain of choice based on the particular fragrance and flavor profile, as well as the specific effects desired.

The Mental and Physical Effects of Terpenes

Although cannabinoids are more typically understood to be responsible for the mental and physical effects of cannabis, terpenes have demonstrated a wide array of such effects as well. See, e.g., Russo, Taming THC: potential cannabis synergy and phytocannabinoid-terpenoid entourage effects, British J. of Pharm., 163:1344-64 (2011).

For example, the terpene myrcene has been shown to have a sedative effect, and is believed to be responsible for a heavy body high, associated with lethargy, lack of motivation, and a reduced ability to function (often referred to as “couch lock”). In fact, certain preparations of myrcene from hops have been employed as a sleep aid. Bisset and Wichtl, Herbal Drugs and Phytopharmaceuticals: A Handbook for Practice on A Scientific Basis, 3rd ed., CRC Press (2004). Furthermore, the effects of myrcene have been demonstrated to be blocked by the drug naloxone, which is also used to block the effects of opioids, especially in overdose. Rao et al., Effect of myrcene on nociception in mice, J. Pharm. Pharmacol. 42: 877-87 (1990).

Beyond myrcene, the terpenes linalool, nerolidol, and pulegone have also shown sedative effects. Others, such as limonene and terpinolene, have by contrast shown stimulating effects. And further others show yet different effects, for example α-humulene acts as an appetite suppressant.

Terpenes may cause effects when consumed because of the modulation of neurotransmitter systems in the brain, as terpenes easily cross the blood-brain barrier. Linalool, for example, has been shown to modulate the glutamate and GABA neurotransmitter systems, and has been demonstrated to possess analgesic, anti-anxiety, anti-inflammatory, and anticonvulsant properties. α-Pinene is an acetylcholinesteral inhibitor, and may thereby aid memory. D-limonene has been shown to exhibit anti-cancer, anxiolytic and immunostimulating properties in humans. β-myrcene, besides the sedative effects noted above, also shows anti-inflammatory, analgesic, and anxiolytic properties. β-caryophyllene possesses anti-inflammatory and gastric cytoprotector activities. Pentacyclic triterpenes, such as β-amyrin and cycloartenol, have been shown to possess various biological activities including anti-bacterial, anti-fungal, anti-inflammatory and anti-cancer properties.

Furthermore, β-caryophyllene, although a terpene, is known to interact with the endocannabinoid system, selectively binding to the CB₂ receptor where it is a functional CB₂ agonist, giving β-caryophyllene an anxiolytic and anti-depressant effect. Through this and other mechanisms, the presence of terpenes in consumed cannabis therefore does not only cause effects individually, but also modifies the effects of the cannabinoids that are present (known as the “entourage effect”). See generally, Russo (2011).

Cannabinoid and Terpene Production in Cannabis Plants

The cannabinoids and terpenes found in cannabis are produced in resin glands known as trichomes. Trichomes, derived from the Greek word ‘trikhoma’ which means ‘growth of hair,’ occur primarily on the plant's flowers (in particular those of the female plant), as well as on the leaves, and to a lesser extent on the stems. Trichomes appear visually as translucent bulbous or mushroom-shaped structures.

It is believed that trichomes evolved as a defense mechanism by cannabis plants, to help protect the plant and its seeds from the dangers of its environment. Physically, the trichomes themselves form a protective layer against offensive insects, preventing them from reaching the surface of the plant. They also help to insulate the plant from high wind and low humidity, and shield against UV-B light rays. Chemically, research shows that the terpenes produced in the trichomes also defend the plant by acting as natural insecticides and fungicides. Indeed, linalool has been adopted for use as a pest control by humans, effective against fleas, fruit flies, cockroaches, and certain moths.

Three primary types of trichomes appear on the cannabis plant: bulbous, capitate-stalked, and capitate-sessile. See Andre et al, Cannabis sativa: The Plant of the Thousand and One Molecules, Front. Plant Sci. 7:19 (2016). Bulbous glands are the smallest (from 15 to 30 micrometers), and are found scattered on the surfaces of aboveground plant parts. Capitate-stalked glands are larger (25 to 100 micrometers) and more numerous than the bulbous glands, and occur predominantly on the female plant's flowers and on the small leaves located near the flowers. Capitate-sessile glands are the largest (reaching a height of 150 to 500 micrometers when their stalks elongate), and appear as the plant matures and begins flowering, primarily on the female flower bracts and on the small leaves that accompany the flowers.

Terpene yield and distribution in the trichomes of cannabis plants varies according to numerous parameters, including the environmental and growing conditions (such as rainfall, temperature, and soil nutrient profile), the maturity of the plant when harvested, and the method of curing and drying the harvested plant, among others.

The Need for Odorless and Terpene-Free Cannabis

To achieve its medicinal and/or recreational effects, cannabis is typically consumed by smoking or vaporization (either of cannabis directly, or of concentrate prepared from cannabis), or by ingestion as part of a cannabis-infused food or drink. (Of course, innumerable other routes of consumption also exist, such as pills, tablets, capsules, bioadhesive particles, wafers, lozenges, drops, chewing gums, tinctures, gels, patches, films, pastes, ointments, creams, lotions, sprays, and aerosols.)

While some cannabis consumers may select cannabis strains based on their aromas and hence their terpene profile, other consumers may prefer to consume a cannabis product that does not have a distinctive cannabis odor or flavor, or indeed any odor or flavor of cannabis at all. For instance, many who choose to consume cannabis by smoking or vaporizing the plant or a plant concentrate, may wish to do so in a discreet way, whether to avoid creating an odor of cannabis during consumption or to avoid having that odor remain after consumption is complete.

Beyond simply seeking an odorless cannabis experience, consumers also may wish to consume terpene-free cannabis to avoid any unwanted effects, such as the sedative effects of β-myrcene, linalool, nerolidol, and pulegone, the stimulating effects of limonene and terpinolene, or the appetite suppressant effects of α-humulene. Depending on the consumer, any or all of these effects may be undesired. Accordingly, eliminating these terpenes from a cannabis product would be of benefit to such consumers.

Also undesired are the effects of δ-3-carene, which has been shown to cause irritation when inhaled, and may be partly responsible for the coughing, itchy throat, and scratchy eyes experienced when smoking cannabis. Eliminating δ-3-carene, therefore, also would be of benefit to consumers.

To consumers that want a consistent, predictable experience each time they consume cannabis, free from the confounding effects caused by terpenes, a terpene-free (i.e., cannabinoid-only) cannabis product likewise would be highly desirable.

For scientists and researchers who want to understand the medicinal effect of cannabinoids when consumed, without any contribution from or interaction with terpenes, a terpene-free cannabis product also would be of great value. (And if wanted, the study of synergies between cannabinoids and single or specific terpenes could be accomplished by adding back to the product just those terpene(s) under consideration.)

Although there is accordingly a real and longstanding need for cannabis products with these characteristics, no satisfactory solutions have been found prior to the present invention.

Solutions that involve attempts to mask or eliminate the fragrance of smoked cannabis have disadvantages or are only partially effective at best. Masking the fragrance typically involves introducing another, stronger, odor, and often only acts to highlight the problem, trading one undesirable odor for another. Eliminating the fragrance typically involves non-portable equipment (e.g., fans and air purifiers) or specialized apparatus (e.g., the odor-eliminating smoking pipe with built in filter and odor absorbing chemicals disclosed in U.S. Pat. App. No. 2011/0240047).

The use of vaporizers and “vape pens” to vaporize or “vape” cannabis and cannabis concentrates has become a popular method of cannabis consumption. With vaporization of cannabis concentrates such as extracts, some of the needs around odor, privacy, and discretion can be satisfied. However, vaporization of cannabis concentrate also has several drawbacks. First, vaping may pose health risks that have not been studied sufficiently or at all. Whereas current evidence does not show that inhalation of cannabis smoke from combustion poses the same health risks caused by inhalation of cigarette smoke (see, e.g., Zhang et al., Cannabis smoking and lung cancer risk: Pooled analysis, Int. J. Cancer 136(4):894 (2015)), there is evidence that inhalation of the vaporization byproducts of cannabis concentrate may pose health risks. This may be due in part to the solvents and carriers present in the concentrates and the vaporization cartridges in which they are used, that are not found in cannabis flower. Recent industry testing has also shown potentially harmful levels of toxic metals such as lead in vaporization cartridges.

Further, many vaporization products contain predominantly or only THC. It has been shown that preparations of THC alone have a poor therapeutic index, and may induce toxic psychosis or cause other adverse reactions when compared to consuming cannabis which contains THC together with other cannabinoids, as when smoking cannabis flower. See Chen, Some of the Parts: Is Marijuana's ‘Entourage Effect’ Scientifically Valid?, Sci. Am. (Apr. 20, 2017). Similarly, because vaping may deliver greater amounts of THC, and may deliver THC in proportions that are high in relation to other cannabinoids (if they are present at all), when compared to the proportions found in cannabis flower, vaping has been reported to lead to increased adverse reactions such as anxiety, paranoia, and nausea. See Spindle et al., Acute Effects of Smoked and Vaporized Cannabis in Healthy Adults, JAMA Network Open, 1(7):e184841 (2018). Moreover, there may be benefits to a product that does not contain terpenes, but that still contains a full spectrum of cannabinoids, given that synergistic (or “entourage”) effects can exist between cannabinoids. See also, e.g., Scott et al, The Combination of Cannabidiol and Δ9-Tetrahydrocannabinol Enhances the Anticancer Effects of Radiation, Mol. Cancer Ther., 13(12):2955-67 (2014); Russo (2011).

Another potential solution to deal with the odors of cannabis consumption is simply to ingest cannabis by way of edibles. However, although the fragrance of edible cannabis products is not as noticeable as the fragrance of cannabis smoke or cannabis flower, consumers of cannabis-infused foods and drinks may also wish to consume products that lack the distinctive odor and flavor of cannabis. For instance, by being able to prepare cannabis edibles without the smell and taste of cannabis, manufacturers can bring other desired smells and tastes to the foreground, as well as create a product that may appeal to a wider audience. And as with vaporized cannabis concentrates, there is still a need for cannabis edibles that lack the odor of cannabis but that still contain a full spectrum of cannabinoids. Cf U.S. Pat. No. 9,629,886 (discussing need for odor- and flavor-free cannabis edibles and beverages, and disclosing concentrated THC and/or CBD products for use in their preparation).

One crucial shortcoming of the consumption of concentrates, whether by vaporization or ingestion, is that many consumers simply prefer to smoke whole cannabis flower because of the ritual and quality of consumption. And in any event, there is an inherent benefit in purely providing to consumers a variety of choices of smokable cannabis products. Cf., e.g., U.S. Pat. App. No. 2018/0352848 (directed to preparing a smokable cannabis-based product with reduced psychoactive effects or possessing a specific cannabinoid and terpene profile).

One hypothetical route to an odorless cannabis plant product would be to select for cannabis strains containing reduced terpene profiles through genetics and breeding. For example, U.S. Pat. No. 9,095,554 discloses and claims cannabis strains containing low levels of the terpene myrcene. The '554 patent states that “samples containing lower relative myrcene contents showed increased positive ratings” in feedback trials with volunteers (id. at col. 15, lns. 15-17) and had effects that were “the most enjoyable” (id. at col. 66, ln. 9). However, while it may be possible to reduce the level of certain terpenes, it is exceedingly difficult to fully eliminate any or all terpenes by breeding alone. And even if such strains would be obtainable by breeding, low terpene chemovars may be less hardy and less able to thrive, because the terpenes also defend the plant by acting as natural insecticides and fungicides. Such a solution also only would be available for a single strain at a time, rather than applicable to cannabis plants generally. Thus, a preferable solution would be to eliminate terpenes after a plant is harvested.

Moreover, a breeding route to an odorless cannabis plant product would likely result in varieties that are concomitantly low in any desired cannabinoids. A significant and positive correlation has been demonstrated between the level of terpenes and cannabinoids produced in cannabis, when grown in standardized conditions. This may be explained by the fact that terpenes and cannabinoids are both synthesized in trichomes. Hence, another route to remove the terpenes from the cannabis plant has remained desirable.

Despite this continued need, to date there have been no known successful solutions that permit the odor and flavor of cannabis to be removed from the plant while retaining a plant end product that maintains both the cannabinoid profile as well as the visual and tactile qualities of the original cannabis flower. These and other characteristics are preserved using the extraction processes of the present invention.

Extraction

Extraction generally refers to various processes of obtaining different natural byproducts from plants. Typically, the goal of an extraction process is to collect certain sought-after compounds, while leaving behind any undesirable compounds and residual plant product. Extraction processes vary from the simple (such as the preparation of tea or coffee from their plant precursors, using heated water) to the complex (involving substantial industrial apparatus and engineering know-how, and multiple controlled parameters). Extraction of plant compounds has been achieved through a number of different methods, such as water or steam distillation, solvent extraction, absolute oil extraction, expression, resin tapping, and cold pressing.

Extraction processes generally are common in the cannabis industry. Such processes are dominated by different forms of solvent extraction, and generally are only concerned with obtaining the cannabinoids and/or terpenes from the cannabis plant, after which the residual plant product is discarded. Such processes are not known to be used to remove terpenes in a manner that leaves a residual plant product that has the qualities discussed herein, and which itself is the desired end-product of the extraction process, rather than a waste product. Accordingly, such previously known processes do not have the benefits of the processes of the present invention.

Distillation

The use of distillation to extract compounds from plants (for instance, to extract essential oils) is generally performed by placing plant material inside a container commonly known as a still. Then, depending on the distillation method, either water or steam is used to separate the volatile constituents (i.e., those capable of being evaporated at the selected temperature and pressure) from the plant material by means of heat.

As the temperature rises in the still, the glands of the plant burst and release their oils and other chemical constituents into the water or steam used in the process. Depending on the temperature, as well as the pressure, time, and other parameters, the volatile constituents rise upward as part of the vapor, depart the still, and enter a collection vessel (the condenser), by means of a connecting pipe. In the condenser, the vapor cools and returns to liquid form, where it is drawn down by gravity, to rest in a collection container known as a receiver or separator. The separator is so-called because the extracted oils do not mix with the recaptured water but typically come to rest on its surface, where they can be siphoned off for collection. (If the extracted constituents additionally consist of any water soluble components, these however will remain in the water fraction, which is termed the hydrosol or hydrolate.) The desired end products from such distillation methods are the extracted oils.

For water distillation (also known as hydrodistillation or “wet” steam distillation), the plant material is placed in direct contact with the water in the still, and is usually fully submerged (by “submerged,” it should be understood that plant material, which in some instances may float, should still be considered submerged as long as sufficient water exists to keep it covered, were it pushed down to the level of the surface of the water). In steam distillation by contrast, the plant material does not come in contact with water, but only with steam. Moreover, steam distillation generally can be performed at higher temperatures, because the extraction of plant materials submerged in liquid cannot be performed at temperatures higher than the temperature at which the liquid boils (e.g., 212° F. for water, at atmospheric pressure), although that temperature can be raised if the still is maintained under pressure.

Solvent Extraction

Other extraction methods for obtaining the terpenes from cannabis also exist. These include supercritical CO₂ extraction and ethanol extraction, as well as the use of various other known solvents, such as butane, propane, and dimethyl ether.

In CO₂ extraction, carbon dioxide, which is a gas at atmospheric pressure, is turned into a supercritical liquid by applying temperature and pressure, so it can be used as a liquid solvent. This solvent is then forced through an extraction vessel packed with finely ground cannabis material (somewhat like how an espresso machine works). This process is repeated until the desired terpenes are extracted. Ethanol extraction works similarly, but with ethanol used as the solvent. See, e.g., U.S. Pat. No. 9,649,349; WO 2016/200438.

However, these and other solvent extraction methods all involve crushing, shredding, chopping, grinding, or otherwise pulverizing the cannabis plant material into a fine powder, and result in a highly-concentrated and much-changed end product as compared to the original plant material. See, e.g., U.S. Pat. Nos. 9,956,498; 9,649,349; 8,445,034.

Hence, none are capable of producing the odorless terpene-free cannabis product that is a primary advantage of the present invention. Additionally, such extraction procedures may leave behind residual solvents and other unwanted toxins. Water and steam distillation avoid these issues with solvent extraction, but neither has before been implemented so as to create an odorless cannabis product. Indeed, despite generalized knowledge and use of various extraction techniques and the teachings demonstrating them, the prior art could not have predicted the benefits that can be achieved by the processes of the present invention.

### BRIEF SUMMARY OF THE INVENTION

It is an object of the present invention to provide processes for preparing an odorless and terpene-free plant product from plant material, comprising contacting the plant material with water or steam at a desired temperature or range of temperatures and for a time which is sufficient to volatilize the odiferous compounds in the plant material, but leave the plant material intact.

Disclosed are suitable apparatus for performing the processes of the invention, such apparatus generally comprising a vessel for holding the plant material, a heating means for boiling water in the vessel or creating steam in the vessel, or a means for introducing steam thereto, means for increasing or decreasing the pressure of the system, and optionally a. condenser to condense the vapor from the vessel, and a container for collecting the condensed liquid.

In disclosed embodiments, the plant material is cannabis plant material, the odiferous compounds are the terpenes found in cannabis plant material, and the odorless plant product is an odorless cannabis plant product.

In further disclosed embodiments, the processes as practiced do not cause pyrolysis or substantial degradation of the plant material, and the resulting cannabis product is obtained without the reduction in the visual and tactile qualities of the plant material.

Additionally, in the disclosed embodiments, the processes produce a cannabis plant product without the loss of cannabinoids as compared to the starting cannabis plant material.

These and other objects, features, and advantages of the present invention may be more clearly understood and appreciated from a review of the following detailed description of the disclosed embodiments and examples, and by reference to the appended figures and claims. The foregoing summary has been made with the understanding that it is to be considered as a brief and general synopsis of only some of the objects and embodiments disclosed herein, is provided solely for the benefit and convenience of the reader, and is not intended to limit in any manner the scope, or range of equivalents, to which the appended claims are lawfully entitled.

### BRIEF DESCRIPTION OF THE FIGURES

Certain aspects of this invention are further described, by way of example only, with reference to the accompanying figures in which:

FIG. 1 is a representation of a hydrodistillation apparatus as could be used in the practice of the processes of the present invention.

FIG. 2 is a representation of a steam distillation apparatus as could be used in the practice of the processes of the present invention, where the steam is generated within the distillation vessel, by heating and boiling water inside the distillation vessel.

FIG. 3 is a representation of a steam distillation apparatus as could be used in the practice of the processes of the present invention, where the steam is generated in a separate boiling vessel, before being introduced into the distillation vessel by injection means.

### DETAILED DESCRIPTION OF THE INVENTION

Among the various aspects of the present invention are odorless plant products, and preferably, odorless cannabis plant products, and processes for their production. While the present invention is described in terms of particular embodiments and applications, it is not intended that these descriptions in any way limit its scope to any such embodiments and applications, and it will be understood that many modifications, substitutions, changes, and variations in the described embodiments, applications, and details of the invention illustrated herein can be made by those skilled in the art without departing from the spirit of the invention, or the scope of the invention as described in the appended claims.

The processes of the present invention comprise steps in which plant material is contacted with heated water or steam, resulting in volitization of the odiferous constituents of the plant material to form a vapor which optionally may be subsequently condensed and removed, leaving behind an odorless plant product. Further, that odorless plant product still contains other constituents of interest, such as cannabinoids, when the processes are performed with cannabis plant material. Suitable apparatus for performing the processes are described below and their use is further illustrated in the accompanying examples.

The Starting Cannabis Plant Material

Herein the term “plant material” refers to the starting material on which the processes are performed, and encompasses whole plants and also parts thereof, for example the aerial parts of the plant or isolated leaves, stems, flowering heads, fruits or roots. With cannabis plant material, it will be understood that the parts used primarily shall be the trimmed buds of the flowering female plant, which contain the greatest density and weight of trichomes and hence contain the greatest amount of terpenes (as well as the greatest amount of cannabinoids). Other plant parts however also shall be able to be advantageously used in the practice of the invention.

While the extraction processes of the invention are particularly preferred for preparing extracts from cannabis plant material, it should be understood that the extraction and removal of terpenes from other types of plants may be performed using the same processes and apparatus, with modifications as could be made in ordinary course. Thus, although reference shall often be made throughout this disclosure to “cannabis plant material” (.or to a “cannabis plant product”), it shall be understood that other plants may also be used.

Any strain of cannabis may be used. However, it should be appreciated that different cannabis strains vary in their terpene profiles, both in terms of the amount of total terpenes present, and in terms of the distribution, type, and amount of individual terpenes. Adjustments can be made to account for such differences, for instance increasing the heat or extending the time of the process for strains with higher total levels of terpenes, or higher percentages of sesquiterpenes and others which are less readily evaporated.

The extraction processes may be carried out starting from freshly harvested plant material, fresh frozen plant material, plant material which has previously been dried and/or cured to remove moisture content, or plant material which otherwise has been subjected to pretreatment. Preferably, the pretreatment shall not result in degradation or deterioration of the plant material, given that one of the benefits of the invention is an end product which maintains the visual and tactile qualities of the starting product. Accordingly, to maximize the benefit of this outcome, it is optimal to start with as good quality of plant material as possible.

Adjustments can also be made to account for pretreatment steps. For example, studies have shown that fresh cannabis plants have an average terpene composition of >90% in monoterpenes and <10% in sesquiterpenes; that proportion changes to around 60/40% respectively, after the plants are dried and cured. This is due to the higher volatility of the monoterpenes, which preferentially evaporate during the drying and curing process. Total terpene content is correspondingly reduced as the monoterpenes evaporate during the drying and curing process. Cannabis plant material has been shown to lose over 30% of its total volatile oil content after drying for a week, and 40-60% after drying for a week followed by storage at room temperature. See Ross and ElSohly, The Volatile Oil Composition of Fresh and Air-Dried Buds of Cannabis saliva, J. Nat. Prod. 59(1):49-51 (1996).

Cannabis plant material that has been dried and cured will also commonly have a larger percentage of terpene molecules that have undergone oxidation to become terpenoids, which are more water soluble. See generally, e.g., Martins et al., Terpenes solubility in water and their environmental distribution, J. Mol. Liquids 241:996-1002 (2017); Martins et al., Selecting Critical Properties of Terpenes and Terpenoids, Ind. Eng. Chem. Res. 56(35):9895-9905 (2017); Fichan et al., Water Solubility, Vapor Pressure, and Activity Coefficients of Terpenes and Terpenoids, J. Chem. Eng. Data 44(1):56-62 (1999).

As discussed above, terpenes are primarily found in the resin glands or trichomes of cannabis plant material. In view of this, prior extraction processes generally use plant material that is crushed, ground up, or otherwise pulverized to break open the trichomes and increase the effective surface area of the plant material, to allow for optimal contact with the water or steam. This may allow for such extraction processes to be run at lower temperatures and for shorter periods, and may result in larger amounts of unoxidized terpenes being collected. However, when pulverized plant material is used for distillation, the resulting plant product, after the distillation is run, is no longer desirable for purposes of consumption by smoking. The processes of the present invention, by contrast, can preferably extract the terpenes from a cannabis plant without the need for such pulverization, and thus result in a plant product that is neither damaged nor degraded, and that retains the visual and tactile qualities of the initial, whole cannabis flower. (Nevertheless, despite the advantages inherent in using whole plant material, pulverized plant material may also be used in the practice of the invention, should the end product not be sought for smoking purposes, but for other purposes, as described further below.)

The Distillation Process

Extraction by distillation works as a process because the compounds obtained are volatile, and hence capable of being vaporized or evaporated and separated from the plant material. Each compound will have a different temperature at which it is volatized, depending on its molecular size (with larger molecules being volatized at higher temperatures) and other factors. By controlling the temperature of a distillation process, therefore, one obtains a specific profile of compounds, that are volatized in that temperature range. For instance, smaller terpenes have already begun to be volatized at room temperatures (as demonstrated by simply smelling cannabis flower), with many further by 100° F., and others at increasingly higher temperatures based on their size. Because of their volatile nature, terpenes are evaporated at temperatures below their boiling points. For example, despite its boiling point of 349° F., limonene will evaporate at 212° F. Cannabinoids, by comparison, begin to be volatized at or closer to their boiling points, with THC generally first at 315° F., and others at higher temperatures (e.g., CBD at 320° F. and CBN at 365° F.) (With figures used herein understood to be at atmospheric pressure, unless otherwise noted).

Generally, differences in vapor pressures dictate how readily the constituent components in the plant material can be separated. As temperature (and negative pressure) are applied, the molecules nearest the surface of the plant material have a tendency to escape into the surrounding atmosphere. With increasing temperature (and decreasing pressure), this tendency increases, and more and more of the molecules volatize. The force generated by these escaping molecules is referred to as the vapor pressure of that component at that particular temperature and pressure. The relative difference in vapor pressures between the different constituent components of the plant material dictates in what manner those components volatize and can be separated.

With these ideas in mind, it should be understood how the distillation processes can also be performed under variations in pressure. Since the volume of the still remains constant, distillation under reduced pressure lowers the temperature at which the components are volatized and extracted. By applying a vacuum, the boiling points and oxygen content are lowered, lessening heat and oxygen exposure. Reducing the temperature of the process thus can prevent oxidation and heat damage to sensitive material. Or, should the temperature be held constant, but the pressure reduced, the run time of the process can be shortened, allowing for more runs in a given period of time. The distillation processes can also be run at increased pressure. With increases in pressure, water boils at higher temperatures. Thus, the submerged plant material in a hydrodistillation process can be heated above 212° F., the boiling temperature at atmospheric pressure. One should readily appreciate that, in choosing how to perform a process, one would consider and weigh tradeoffs between time, temperature, and pressure to determine the optimal parameters (considering as well factors such as the desired plant product, optional terpene collection, electricity costs, operator costs, and the like).

The Resulting Cannabis Plant Product

The resulting dried cannabis plant product is an odorless and terpene-free product that can be consumed directly by smoking or vaporization, ground and used in pre-rolls (or used in pre-rolls directly, if processed pre-ground), incorporated into edibles or beverages, further extracted to obtain the residual cannabinoids, or utilized in any other number of advantageous ways.

By “odorless” it generally should be understood that the resulting plant product would have no discernable odor of cannabis to a neutral untrained observer (i.e., one who would be familiar with the fragrance of cannabis and cannabis smoke, but would not be an expert in distinguishing between odors or in the odors of cannabis). For example, a cannabis user selecting a cannabis plant product for consumption, when odorless refers to whole cannabis flower. Odorless may also refer to a plant product which does not produce the characteristic fragrance of cannabis when consumed by combustion (i.e., when smoked), similarly when judged by a neutral untrained observer. It should be understood that odorless does not mean without any odor whatsoever, but rather without any odor of cannabis. Consequently, for example, the smoke of the plant product is odorless when it does not smell like cannabis, but it may still be recognizable as being smoke from combustion.

One should also understand that, to be odorless, only the odiferous terpenes that contribute to the fragrance of cannabis and cannabis smoke need be removed. Over 20,000 varieties of terpene molecules exist, of which at least 120 can be found in cannabis. However, not all terpenes produce an odor detectable to a human observer. Accordingly, an “odorless” cannabis plant product need not be measurably free from all terpenes to fall within the meaning of the invention (although generally, all volatile terpenes will nonetheless be removed).

Olfactory testing by human subjects has been used in the scientific literature to verify odors, specifically with cannabis. For instance, in Ross and ElSohly (1996), the investigators had subjects familiar with the smell of cannabis identify and evaluate the smell of different cannabis preparations. The investigators asked whether the subjects were able to recognize the smell of those preparations as being that of cannabis. And Courts have held that the smell of cannabis coming from a vehicle at a traffic stop provides probable cause to justify a search of the vehicle, and that the smell of cannabis on a person provides probable cause to arrest and search that person, based on the olfactory judgment of a police officer.

A plant product may be considered “terpene-free” if it can be shown to have no detectable terpenes, as tested by generally accepted laboratory procedures, such as those used by Steep Hill Labs, Inc., discussed further below. “Terpene-free,” generally should be understood to mean without any odiferous terpenes, as discussed above. “Terpene-free,” where so specifically defined, may also mean without the particular characteristics of a distinct terpene profile (for instance if certain terpenes only are selectively extracted) or without any other particular terpene characteristics.

Importantly, “terpene-free,” within the meaning of the invention, need not require that a plant product be entirely free from all measurable terpenes. “Terpene-free” may also mean, where so defined, that no terpenes exist below a selected detectability threshold. Although there are over 120 different types of terpenes found in cannabis, for any given strain only a handful are generally produced at measurable levels. For instance, the terpene tests performed by Steep Hill Labs only test for ten of the most common terpenes found in cannabis (i.e., (β-caryophyllene, caryophyllene oxide, citronellol, α-humulene, D-limonene, linalool, β-myrcene, phytol, α-pinene, and terpinolene.) Testing for, and the absolute elimination of, every single type of possible terpene (or of every single terpene molecule for any given type) is neither envisioned nor necessary to achieve the benefits of the invention. For one, not all terpenes need be eliminated for a plant product to be considered “odorless.” Further, terpenes are generally considered to be “pharmacologically relevant” when present in concentrations of at least 0.05% in plant material. See Hazekamp and Fischedick, Metabolic fingerprinting of Cannabis sativa L., cannabinoids and terpenoids for chemotaxonomic and drag standardization purposes, Phytochem. 2058-73 (2010); Russo, Taming THC (2011). Thus, a “terpene-free” plant product may properly include those defined to mean a plant product having no terpenes present at “pharmacologically relevant” levels, or at other detectability thresholds.

Broadly, it should be understood that “odorless” and “terpene-free” are simply two ways of understanding and confirming the same general characteristics of the plant product; that is, one through human perceptive means, and one by laboratory testing. It should be appreciated that, generally speaking, an “odorless” plant product has been rendered such because it has been made “terpene-free,” and conversely, a “terpene-free” plant product will be “odorless.” Where a plant product has been determined to be “terpene-free,” it accordingly shall also be considered a demonstration that it is “odorless,” and vice-versa, although specific reference to one or the other term shall be still made where useful herein.

It also should be appreciated that besides the characteristic of being odorless, the plant product of the processes of the invention carries other benefits such as producing a smoke which is smoother, “cleaner,” and less harsh, in part due to the reduction in total chemical content. Further, although preferably all plant material used in the processes should be free from any impurities such as mold or fungus, it is recognized that the processes may reduce or eliminate certain such impurities in the plant material, should they exist.

Lastly, and as described further above, it should be appreciated that another significant benefit of the present invention is the ability to obtain odorless and terpene-free plant product in the form of intact whole cannabis buds that are neither damaged nor degraded, and that retain the visual and tactile qualities of the initial plant material.

Detailed Operation of the Distillation Processes

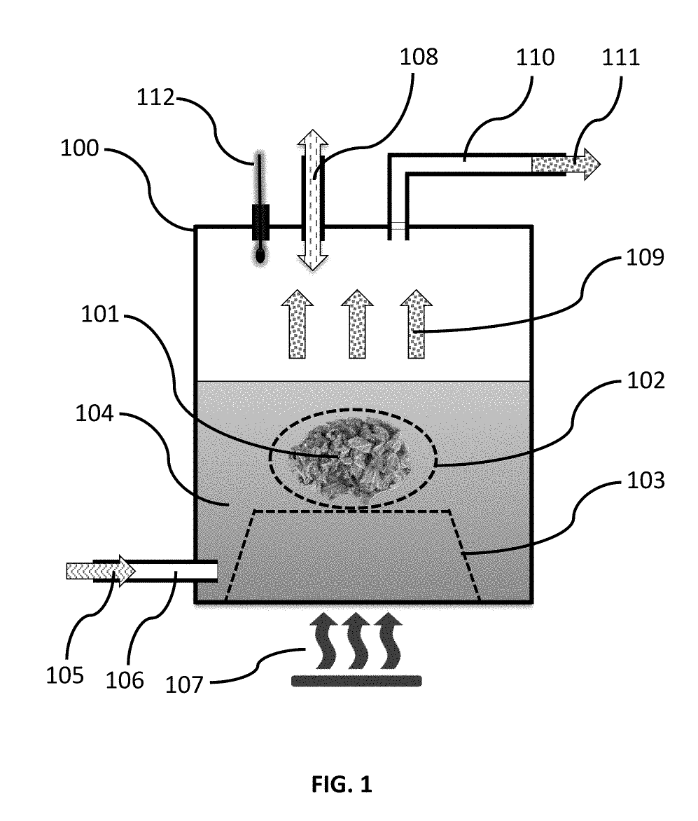

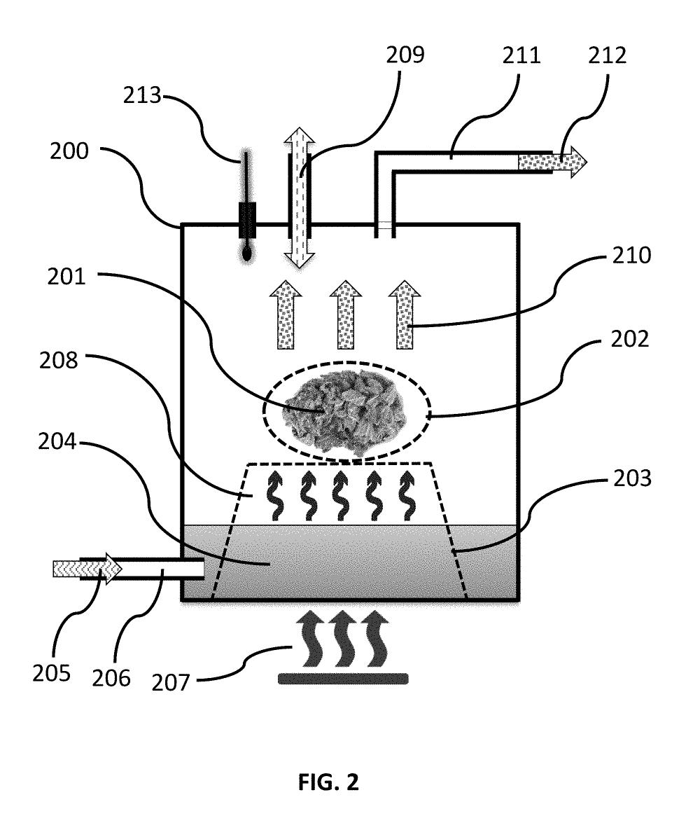

The following description details the steps used to perform embodiments of the distillation processes, with reference to FIGS. 1, 2, and 3.

Hydrodistillation

A hydrodistillation process is performed using a distillation vessel 100. The distillation vessel should be of sufficient size to contain the plant material (and any containing and/or supporting means) and the volume of water necessary to perform the process. Suitable sizes generally range from 5 L to 50 L, but smaller sizes of if or 2 L would be suitable for running test batches or for small home kits, and larger sizes over 50 L would be suitable for large commercial runs.

Reference may be made to the amount of plant material to be used in the process to determine the volume of water necessary, and thus the distillation vessel size. Batch sizes may generally range from as little as a gram, to as much as several pounds or more. When performing a hydrodistillation process, the distillation vessel should contain sufficient water to keep the plant material fully submerged throughout the process. Accordingly, the size of the distillation vessel at minimum should be large enough to contain enough water to cover the plant material until further water is added (if water is added during the process, to account for water lost as steam), or should be large enough to contain sufficient water to both cover the plant and keep it submerged throughout the entire process (if water is not added during the process, to replace the water lost as steam). If a containing means is used to hold the plant material inside the distillation vessel, the vessel size should be sufficient to enclose the containing means and allow for the plant material within the containing means to be fully submerged in the water. With these guidelines in mind, one of ordinary skill will appreciate that the process is readily scaled and that the choice of distillation vessel is a function of the process scale.

In a first step, the plant material selected for the process is placed inside the distillation vessel. The plant material 101 may be placed directly within the distillation vessel. Alternately, the plant material is first placed within a containing means 102, and then the containing means is placed within the distillation vessel.

Suitable containing means for holding the plant material within the vessel include partially and fully enclosed containers that hold the plant material in place but allow water to pass through, contact the plant material, and circulate within the distillation vessel. Suitable such containing means include colanders, strainers, baskets, infusers, and the like. Such containing means may be made out of plastic, metal, cloth, or other suitable materials. One would understand that numerous means could satisfactorily be employed, keeping in mind the goal of maintaining the plant material contained and fully submerged, while allowing water to circulate and pass through, and allow the plant compounds as would be removed from the plant material to pass out. Depending on the shape and size of the vessel, multiple containing means can be used together, for instance permitting different portions of plant material to be kept together or kept within the distillation vessel for different lengths of time.

In an additional embodiment, the plant material may be placed on top of a supporting means 103 inside the distillation vessel, such as a wire or mesh rack, a grate, a screen, or the like, which supports the plant material and keeps it raised away from the bottom surface of the distillation vessel, while at the same time still allows it to remain fully submerged throughout the process. If the distillation vessel is heated by contact between the vessel bottom and a heat source, such supporting means may be advantageously used to keep the temperature of the plant material consistent, by keeping it away from contact with the vessel bottom, which may be of higher temperature than the average water temperature in the vessel. The plant material may also be placed first within a containing means, and the containing means then placed on the supporting means, allowing for the benefits of both to be obtained.

In a second step, water is introduced into the distillation vessel. Water 104 may be added manually into the distillation vessel, or water 105 may be added through a connected water line 106. The distillation vessel is filled with sufficient water to cover the plant material throughout the process. That is, enough water must be used to account for the amount of water lost as steam during the process, so that the plant material remains submerged throughout the process, and so that the plant material does not come to rest on the bottom of the vessel at any point during the process where it could become scorched or damaged by heat. Additional water may be added during the process to account for the water lost as steam, returning to and repeating the second step one or more times after the water has been heated. If the water in the vessel is replaced during the process, it should be understood that methods for replacing the water are preferable where they have the least effect on the temperature and pressure of the system (such as replacement with boiling or heated water versus cold water, which would lower the temperature of the total water and extend the time of the process, or replacement using a connected water line 106 or other means that does not open the system resulting in a change in pressure). Although the total amount of water used will vary depending on the time, temperature, and pressure of the process, it shall be determinable in the ordinary course of running the process, either through trial or by calculation using the parameters of the system.

It should be apparent that the first and second steps could be reversed (i.e., water could be placed within the vessel first, and then the plant material added) without affecting the process.

In a third step, the water in the distillation vessel is heated and brought to the desired temperature. The distillation vessel is heated using heating means 107 such as external gas and electric heating elements, immersion heating elements, thermal fluid systems, jacketed or circulation heaters, and the like. Temperature sensors, gauges, and controls may be advantageously used to monitor the temperature, and the heat may be manually or automatically reduced or turned off once the desired temperature is reached.

In a fourth step, the temperature of the distillation vessel is maintained at a desired temperature or range of temperatures until the chosen process time is reached. Alternatively, a process can be run using two or more distinct temperature stages, which may be used for selective extraction of different constituents. Temperature sensors, gauges, and controls may be advantageously used to monitor and manually or automatically maintain the temperature throughout the process. Manual temperature control would include taking periodic temperature readings using for example inline or probe-type thermometers as in 112, and manually adjusting the heating means during the process accordingly. Alternatively, such a thermometer 112 could be operatively coupled to the heating means 107 to adjust the temperature automatically. Equipment could also be used to monitor and maintain the temperature automatically, based on pre-set parameters.

The temperature and time of the process shall be chosen so as to permit the terpene content of the plant material to be sufficiently volatized and removed. The terpene content of the plant material will be understood to be sufficiently volatized and removed when the resulting plant product is odorless and terpene-free as desired. It should be understood that the process time is a function of the temperature, the vessel size, the vessel pressure, the amount of plant material undergoing the process, and the specific strain and harvest batch of plant material being used. For example, at lower temperatures a longer period of time will be taken for complete extraction of a given amount of terpenes. The optimum temperature and time will be understood to vary depending on the profile and amount of terpenes in the plant material. As an example, heavier molecular weight terpenes will require greater temperatures to extract. As another example, a plant material with a greater amount of terpenes generally will require greater temperatures (and/or longer process times) for an extraction to run to completion. Importantly, the temperature of the process shall be consistently maintained below that temperature at which any constituents that are desired to remain in the resulting plant product (such as cannabinoids) are volatized.

By way of example, temperatures in the range of 140-300° F. are observed to result in preferential volatilization of terpenes from most cannabis plant material. Generally, it should be appreciated that for cannabis plant material temperatures shall be under 350° F., and preferably shall be under 300° F., to avoid the volatization and loss of cannabinoids, and to not cause substantial pyrolysis (i.e., decomposition brought about by high temperatures) of the plant material. Under atmospheric or increased pressure, temperatures between 212-300° F. should be considered preferred for cannabis plant material, whereas temperatures between 140-212° F. (at reduced pressure) should be considered preferred to minimize the thermal degradation of the extracted terpenes, if they are intended to be collected and saved.

One of skill in the art will, moreover, appreciate that the optimum temperature and time may vary depending on the characteristics of the apparatus used to carry out the process, for example the amount of raw material processed in each batch and the amount of contact it has with the extracting water or steam. Thus, as with any given plant material, for any given system, temperature and time likewise would be understood to be optimized either mathematically or empirically. But in any process, the key indicator of sufficient run time is when the desired end point is reached, namely, when the plant product is rendered odorless and terpene-free. Generally, it is observed that for temperatures in the range of 140-300° F., process times of between one and four hours are typically sufficient to remove the volatile terpenes from the plant material, without resulting in substantial loss of cannabinoids or degradation of its visual and tactile quality.

Typically, the distillation vessel shall be covered or sealed in a manner allowing for the pressure within the vessel to remain constant and controlled. Pressure sensors, gauges, and controls may be advantageously used to monitor and manually or automatically maintain the pressure throughout the process. If the distillation vessel is to be maintained under negative or positive pressure, additional equipment for that purpose may be added, with such equipment connected to the distillation vessel to adjust the pressure 108. To allow the steam carrying the volatized terpenes 109 to escape during the process, the distillation vessel may be partially or loosely covered or a steam control valve or pressure relief valve may be used, or any other such steam exit pathway 110. Optionally, a separate condenser apparatus may be attached, which additionally has the benefit of collecting the evaporated and vaporized terpenes 111 as they exit the distillation vessel.

In a fifth step, the plant material in the distillation vessel 100 is removed. If needed, the distillation vessel or other parts of the apparatus first may be allowed to sufficiently cool before removing the plant material.

Steam Distillation

A steam distillation process is performed with many similarities to the hydrodistillation process described above, but with a few key differences, described below.

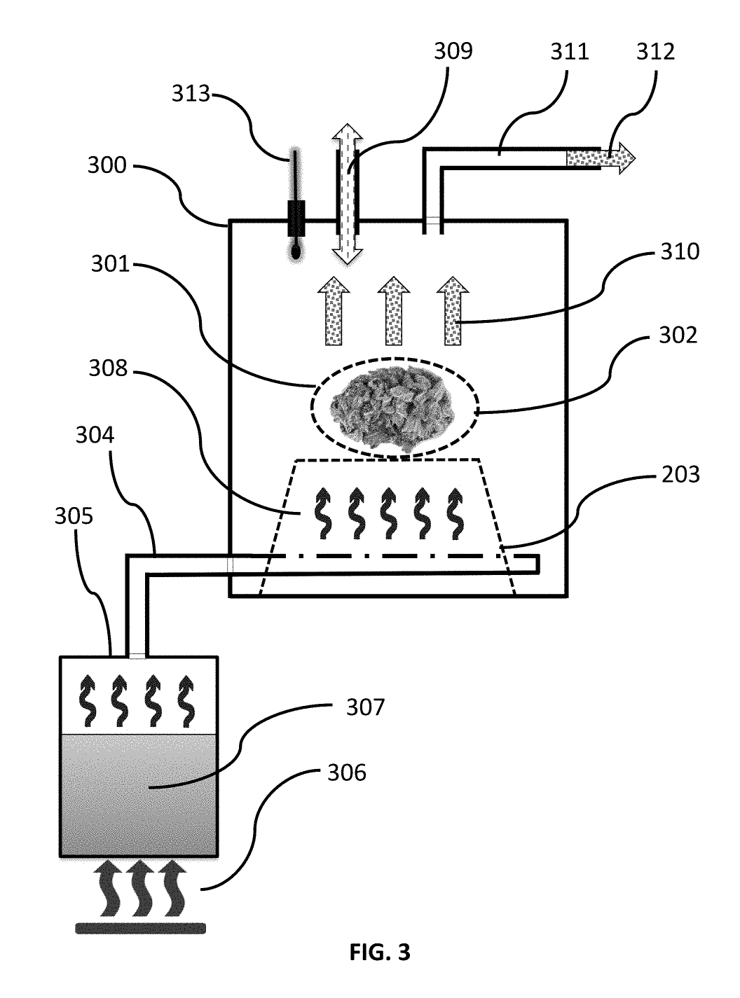

As with a hydrodistillation process, a steam distillation process is performed. using a distillation vessel 200, or as shown in 300 when steam injection means is used. The distillation vessel 200, 300 should be of sufficient size to contain the plant material, and any containing and/or supporting means. The distillation vessel 200, when the steam is generated within the distillation vessel itself, should be of sufficient size to contain the volume of water necessary to perform the process. If steam injection means is used to introduce steam into the distillation vessel 300, then the distillation vessel accordingly should be of sufficient size to contain the injection means, together with the plant material and any containing and/or supporting means; see also FIG. 3 and discussion below. Suitable sizes of distillation vessels 200, 300 generally range from 5 L to 50 L, but smaller sizes of 1 L or 2 L would be suitable for running test batches or for small home kits, and larger sizes over 50 L would be suitable for large commercial runs. As with a hydrodistillation process, one will appreciate that a steam distillation process is readily scaled and that the choice of distillation vessel is a function of the process scale, keeping the above guidelines in mind.

In a first step, the plant material 201, 301 selected for the process is placed inside a containing means 202, 302 and/or on top of a supporting means 203, 303 within the distillation vessel, so that the plant material is kept suspended above the water 204 or above the steam injection means 304 throughout the process. Suitable containing means for suspending the plant material within the vessel include partially and fully enclosed containers that hold the plant material in place but allow steam to pass through, such as colanders, strainers, baskets, infusers, and the like. The supporting means may be a wire or mesh rack, a grate, a screen, or the like, that permits steam to pass through. Such containing and supporting means may be made out of plastic, metal, cloth, or other suitable materials. One would understand that numerous containing and supporting means could satisfactorily be employed, keeping in mind the goal of maintaining the plant material in place but allowing steam to pass through, along with the plant compounds as would be removed from the plant material. Depending on the shape and size of the distillation vessel, multiple containing and/or supporting means can be used together, such as multiple racks mounted vertically, or multiple baskets mounted laterally, or multiple baskets placed laterally on multiple racks, permitting a greater amount of plant material to be used in the process, and greater control over process times and degree of steam contact for different plant material.

In a second step, water is heated to generate steam. In one embodiment, the distillation vessel 200 shall be filled at the outset with sufficient water so that the process can run to completion before the evaporation of the water in the vessel is complete, understanding that the amount of water shall depend on the time, temperature, and pressure of the process. In an alternate embodiment, the water in the distillation vessel 200 can be replaced as the process runs, with water 205 entering the distillation vessel through connecting means 206, and as described more fully above for a hydrodistillation process. In such embodiments, the water in the distillation vessel is heated and brought to the desired temperature. The distillation vessel is heated using heating means 207 such as external gas and electric heating elements, immersion heating elements, thermal fluid systems, jacketed or circulation heaters, and the like. Temperature sensors, gauges, and controls may be advantageously used to monitor the temperature and maintain the temperature so as to generate the desired amount of steam.

In a further alternate embodiment of a steam distillation process, steam injection means 304 are used to introduce steam 308 into the distillation vessel 300. The steam is generated from water heated in a separate boiling vessel 305, using heating means 306 such as for the distillation vessel, which boils the water 307 in the boiling vessel to generate steam. That steam then passes through the injection means 304 and exits as steam 308 into the distillation vessel 300. It should readily be appreciated that using steam injection means permits a process to be run without concern for the amount of water remaining within the distillation vessel at any given time point, or the need to refill the distillation vessel during the process (however, one would understand that the boiling vessel must be maintained with water).

If a steam distillation process is performed using steam injection means, it also would be appreciated that repeated runs of a process can be easily performed, by replacing the plant material after the process time has been reached, and running the process again. In this manner, the process can be run nearly continuously, as long as water is maintained in the boiling vessel (which may be done by connecting the boiling vessel directly to a water line, for instance).

In a third step, the plant material in the distillation vessel is contacted with the steam 208 generated by boiling the water in the distillation vessel, or the steam 308 introduced into the distillation vessel by the steam injection means.

As with a hydrodistillation process, the distillation vessel during a steam distillation process shall be covered or sealed in a manner allowing for the pressure within the vessel to remain constant and controlled. Pressure sensors, gauges, and controls may be advantageously used, and if the distillation vessel is to be maintained under negative or positive pressure, additional equipment for that purpose may be added, with such equipment connected to the distillation vessel to adjust the pressure 209, 309. To allow the steam carrying the volatized terpenes 210, 310 to escape during the process, the distillation vessel may be partially or loosely covered or a steam control valve or pressure relief valve may be used, or any other such steam exit pathway 211, 311. Optionally, a separate condenser apparatus may be attached, which additionally has the benefit of collecting the evaporated and vaporized terpenes 212, 312 as they exit the distillation vessel.

In a fourth step, the temperature of the system is maintained at a desired temperature or range of temperatures until the chosen process time is reached. Alternatively, a process can be run using two or more distinct temperature stages, which may be used for selective extraction of different constituents. Temperature sensors, gauges, and controls may be advantageously used to monitor and manually or automatically maintain the temperature throughout the process. Manual temperature control would include taking periodic temperature readings using for example inline or probe-type thermometers as in 213, 313, and manually adjusting the heating means during the process accordingly. Alternatively, such a thermometer 213, 313 could be operatively coupled to the heating means 207, 306 to adjust the temperature automatically. (An additional heating means may also be added to the distillation vessel 300 for the purpose of temperature control.) The temperature and time of a process shall be chosen so as to permit the terpene content of the plant material to be sufficiently volatized and removed, as discussed in detail above.

In a fifth step, the plant material in the distillation vessel 200, 300 is removed. If needed, the distillation vessel or other parts of the apparatus first may be allowed to sufficiently cool before removing the plant material.

Post-Distillation Steps

The resulting plant product may be dried using methods such as those used for drying post-harvest plant material, for example by hanging the plant product, or by placing the plant product on a drying means such as a mesh, rack, perforated shelf, or the like, in an appropriate environment. Preferably, fans would be used to circulate air around and through the plant product as it dries, and the plant product would be rotated by hand periodically, to ensure consistent drying and the preservation of the visual and tactile qualities of the plant. With pre-ground plant material, it is critical to ensure the drying means is of appropriate size so that it does not allow any loss of the plant material.

Process Variations

Preferably, the plant material is not ground up or otherwise altered before use in the process, so as to preserve its shape and visual and tactile appeal as the final product. However, in a variation on the process, the plant material may be ground to the desired coarseness prior to being placed in the containing means or on the supporting means. While whole plant material should be considered preferable to generate the final cannabis plant product, if odorless ground flower is the desired final plant product (for instance, to use in creating cannabis “prerolls”), it may be appreciated that using preground plant material may allow for faster process times compared to starting with whole plant material.

In a second variation on the process, rather than either whole plant product or ground plant product, a pulverized or very finely ground product may be utilized. Use of such a finely ground product may further reduce the process times and temperatures, and may be useful for obtaining a product which may subsequently used in other extraction methods (e.g., oil or solvent extraction) to remove the cannabinoids. Such a resulting product is an odorless and taste-free extract that can be incorporated into edibles or beverages, used for extract products such as wax pens (oil cartridges), lotions and topicals, or utilized in any other number of advantageous ways. One would appreciate that modifications to the process may be necessary to properly hold the finely ground product during distillation (e.g., a finer mesh containing and/or supporting means), and to hold the finished product during drying (e.g., a finer mesh drying means).

In other variations, a gas other than atmospheric air (e.g., pure nitrogen or another inert gas, or a blend of gases) may be used inside the distillation unit, which can prevent or retard the oxidation of compounds by eliminating or reducing the presence of oxygen.

Process Apparatus

Suitable apparatus for the practice of the invention can be created from separate off-the-shelf components, or a complete hydrodistillation or steam distillation system could be purchased and modified as necessary.

Preferably, the apparatus should be made from stainless steel, glass, or copper components, or from non-reactive materials such as ceramic or enamel coated metal. Materials should be able to withstand the temperatures and pressures used in the chosen processes.

The apparatus described for the processes of the invention should be understood to comprise merely the minimum equipment necessary to run the processes successfully. Other equipment could be added based on the needs and desires of the operator, and indeed, the addition of other equipment to aid in the operation of the processes should be understood to be both envisioned and within the scope of the invention. For instance, it is envisioned that one may add, among numerous other modifications, additional tubing and connection means either into the system (e.g., water or steam lines) or out of the system (e.g., steam lines or condenser lines), automatic water level or flow rate controllers, low water level shut off means, automatic vacuum and pressure controllers, liquid, vacuum, or discharge pumps, additional heating or chilling units, external condensers and cold traps, product collection and sampling receivers, digital computer or programmable logic controllers, electronic control panels, and other instrumentation means.

It should be understood that the process steps of the invention are open in that other steps may be added between the steps or subsequent to them without departing from the spirit of the invention. For instance, the vapor produced is also condensed and collected. The condensate may be a homogeneous liquid or may, depending on the nature of the starting plant material, form a mixture of oily and aqueous components. In the latter case, the apparatus used for carrying out the process may further include means for collecting the condensate in two or more separate fractions. The terpene-rich fraction may also have a commercial value, for instance as oils for aromatherapy or to be added back into other cannabis products.

The processes of the invention exhibit markedly increased selectivity for extraction of terpenes over cannabinoids. Using these processes it is thereby possible to retain a cannabinoid-rich cannabis plant product which is odorless and terpene-free. This is further elucidated by reference to the following examples.

### EXAMPLES

To demonstrate the effectiveness of the invention, cannabis plant material was obtained for testing. The cannabis plant material, obtained together and from a single strain (Kali Mist), was divided into five equivalent portions measuring one eighth ounce each. A first portion was set aside for testing as a control sample. The remaining four portions were used to perform four runs of the processes of the invention (below, as Examples 1-4), and subsequently submitted for testing.

### Example 1

In a first example, a hydrodistillation process was practiced, such as disclosed above. Dried and unground cannabis plant material (i.e., intact whole trimmed buds) was placed within a five liter stainless steel distillation vessel, and sufficient water was added to the vessel to fully cover the plant material. The plant material was maintained submerged in a loose fashion underneath the water level, and was not contained in any separate containing means. The vessel was then loosely covered, to allow for steam to escape. The temperature of the water in the vessel was raised, by contact between the vessel and a gas cooktop. The water in the vessel was brought to a boil. After 30 minutes, the cover of the vessel was removed, to ensure that there was still sufficient water in the vessel to cover the plant material, and more water was added to ensure that the plant material would remain submerged during another 30 minute session.

In total, three 30-minute sessions were performed, with the water checked and supplemented following each. Subsequently, two 40-minute sessions were run, with the water similarly checked and supplemented in between, so that a total of five sessions were performed (three 30-minute and two 40-minute, with four water checks). The vessel was kept loosely covered so that steam could escape the vessel throughout (the steam was not collected, but was allowed to escape into the atmosphere)

At the end of the five sessions, the cannabis plant material was removed. After removal, the resulting cannabis plant product was placed on nylon mesh netting where it was left to dry for 24 hours, in a dark environment kept at room temperature, and with box fans used for air circulation. After drying, the plant product was moved to a plastic container, in which it was transported to a testing facility within 24 hours.

The resulting cannabis plant product was submitted for testing and analysis to Steep Hill Labs, Inc., at 1005 Parker Street, Berkeley, Calif. 94710 (www.steephill.com),

Steep Hill Labs analyzed the Example 1 plant product for terpene content. A certificate of analysis was obtained for a terpene test of the product (Sample identification BK12224-2), which reported the terpene compound profile in Table 1 below:

TABLE 1

| Terpene | % | mg/g |
|---|---|---|
| β-Caryophyllene | Not Detected | |
| Caryophyllene oxide | Not Detected | |
| Citronellol | Not Detected | |
| α-Humulene | Not Detected | |
| D-Limonene | Not Detected | |
| Linalool | Not Detected | |
| β-Myrcene | Not Detected | |
| Phytol | Not Detected | |
| α-Pinene | Not Detected | |
| Terpinolene | Not Detected | |

Steep Hill Labs also analyzed the Example 1 plant product for cannabinoid content. A certificate of analysis was obtained for a standard potency test of the product (Sample identification BK 12224-2), which reported the cannabinoid compound profile in Table 2 below:

TABLE 2

| Cannabinoids | % | mg/g |
|---|---|---|
| THCA | 0.27 | 2.7 |
| THC | 2.9 | 29.0 |
| CBDA | Not Detected | |
| CBD | Not Detected | |
| CBG | Not Detected | |
| CBN | 0.071 | 0.71 |

Decarboxylated cannabinoid values for the Example 1 plant product were also determined by Steep Hill Labs, as 3.1% for THC (Obtained by calculating THCA×0.88+THC), and as “Not Detected” for CBD (obtained by calculating CBDA×0.88+CBD),

### Example 2

In a second example, a hydrodistillation process was practiced in primary respects similar to that of Example 1, however a single session of 2.5 hours was performed, instead of multiple sessions with additional water added in between. This permitted the maintenance of a more consistent temperature and pressure in the vessel, without intermittent temporary reductions due to opening the vessel for the replacement of boiling water with water at a lower temperature.

The resulting cannabis plant product was subsequently dried as above, and then submitted for testing and analysis to Steep Hill Labs. As with the plant product of the above example, Steep Hill Labs also analyzed the Example 2 plant product for terpene content. A certificate of analysis was obtained for a terpene test of the product (Sample identification BK12225-2), which reported the terpene compound profile in Table 3 below:

TABLE 3

| Terpene | % | mg/g |
|---|---|---|
| β-Caryophyllene | Not Detected | |
| Caryophyllene oxide | Not Detected | |
| Citronellol | Not Detected | |
| α-Humulene | Not Detected | |
| D-Limonene | Not Detected | |
| Linalool | Not Detected | |
| β-Myrcene | Not Detected | |
| Phytol | Not Detected | |
| α-Pinene | Not Detected | |
| Terpinolene | Not Detected | |

Steep Hill Labs also analyzed the Example 2 plant product for cannabinoid content. A certificate of analysis was obtained for a standard potency test of the product (Sample identification BK12225-2), which reported the cannabinoid compound profile in Table 4 below.

TABLE 4

| Cannabinoids | % | mg/g |
|---|---|---|
| THCA | 0.21 | 2.1 |
| THC | 3.5 | 35.0 |
| CBDA | Not Detected | |
| CBD | Not Detected | |
| CBG | Not Detected | |
| CBN | 0.063 | 0.63 |

Decarboxylated cannabinoid values for the Example 2 plant product were also determined by Steep Hill Labs, as 3.7% for THC, and as “Not Detected” for CBD (obtained using the same calculations as above).

### Example 3

In a third example, a single 2.5 hour steam distillation process was performed, with the cannabis plant material suspended above the water level in the distillation vessel rather than being submerged. The cannabis plant material was placed as whole trimmed buds (i.e., not ground or pulverized) into a porous metal strainer that sat above the water line and allowed for steam to pass through, but retained all of the plant material inside during the process.

The resulting cannabis plant product was subsequently dried as above, and then submitted for testing and analysis to Steep Hill Labs. As with the plant product of the above examples, Steep Hill Labs also analyzed the Example 3 plant product for terpene content. A certificate of analysis was obtained for a terpene test of the product (Sample identification BK12226-2), which reported the terpene compound profile in Table 5 below:

TABLE 5

| Terpene | % | mg/g |
|---|---|---|
| β-Caryophyllene | Not Detected | |
| Caryophyllene oxide | Not Detected | |
| Citronellol | Not Detected | |
| α-Humulene | Not Detected | |
| D-Limonene | Not Detected | |
| Linalool | Not Detected | |
| β-Myrcene | Not Detected | |
| Phytol | Not Detected | |
| α-Pinene | Not Detected | |
| Terpinolene | Not Detected | |

Steep Hill Labs also analyzed the Example 3 plant product for cannabinoid content. A certificate of analysis was obtained for a standard potency test of the product (Sample identification BK12226-2), which reported the cannabinoid compound profile in Table 6 below.

TABLE 6

| Cannabinoids | % | mg/g |
|---|---|---|
| THCA | 0.23 | 2.3 |
| THC | 3.0 | 30.0 |
| CBDA | Not Detected | |
| CBD | Not Detected | |
| CBG | Not Detected | |
| CBN | 0.062 | 0.62 |

Decarboxylated cannabinoid values for the Example 3 plant product were also determined by Steep Hill Labs, as 3.2% for THC, and as “Not Detected” for CBD (obtained using the same calculations as above).

### Example 4

In a fourth example, a single four hour steam distillation process was performed. As in Example 3, the cannabis plant material was suspended above the water level in the distillation vessel, by being placed as whole trimmed buds into a porous metal strainer.

The resulting cannabis plant product was subsequently dried as above, and then submitted for testing and analysis to Steep Hill Labs. As with the plant product of the above examples, Steep Hill Labs also analyzed the Example 4 plant product for terpene content. A certificate of analysis was obtained for a terpene test of the product (Sample identification BK12227-2), which reported the terpene compound profile in Table 7 below:

TABLE 7

| Terpene | % | mg/g |
|---|---|---|
| β-Caryophyllene | Not Detected | |
| Caryophyllene oxide | Not Detected | |
| Citronellol | Not Detected | |
| α-Humulene | Not Detected | |
| D-Limonene | Not Detected | |
| Linalool | Not Detected | |
| β-Myrcene | Not Detected | |
| Phytol | Not Detected | |
| α-Pinene | Not Detected | |
| Terpinolene | Not Detected | |

Steep Hill Labs also analyzed the Example 4 plant product for cannabinoid content. A certificate of analysis was obtained for a standard potency test of the product (Sample identification BK12227-2), which reported the cannabinoid compound profile in Table 8 below.

TABLE 8

| Cannabinoids | % | mg/g |
|---|---|---|
| THCA | 0.21 | 2.1 |
| THC | 2.5 | 25.0 |
| CBDA | Not Detected | |
| CBD | Not Detected | |
| CBG | Not Detected | |
| CBN | 0.046 | 0.46 |

Decarboxylated cannabinoid values for the Example 4 plant product were also determined by Steep Hill Labs, as 2.7% for THC, and as “Not Detected” for CBD (obtained using the same calculations as above).

The initial cannabis plant product, which was retained as a control sample, was also submitted for testing and analysis to Steep Hill Labs. The results from the Steep Hill Labs analyses of the cannabis plant product from each of Examples 1-4 were thus able to be compared against this control sample.

As with the plant product of the above examples, Steep Hill Labs also analyzed the control sample for terpene content. A certificate of analysis was obtained for a terpene test of the control sample (Sample identification BK12228-2), which reported the terpene compound profile in Table 9 below:

TABLE 9

| Terpene | % | mg/g |
|---|---|---|
| β-Caryophyllene | Not Detected | |
| Caryophyllene oxide | Not Detected | |
| Citronellol | Not Detected | |
| α-cumulene | Not Detected | |
| D-Limonene | 0.033 | 0.33 |
| Linalool | Not Detected | |
| β-Myrcene | 0.43 | 4.3 |
| Phytol | Not Detected | |
| α-Pinene | 0.081 | 0.81 |
| Terpinolene | 0.028 | 0.28 |

The control sample (Sample identification 111467918356) was also analyzed by Steep Hill labs for cannabinoid content. Steep I=1ill Labs provided a verified cannabis test report showing a raw (pre-decarboxylation) THC-A and THC content of 4.6% and CBD-A and CBD content of <2%. The test report also showed a heated (post-decarboxylation) THC content of 3.5% and CBD content of <2%.

By comparing the results of the terpene analyses from Examples 1-4 in Tables 1, 3, 5, and 7 to the control sample result in Table 8, it is demonstrated that the terpene content of the resulting cannabis plant product is removed from the initial cannabis plant material; indeed, no terpenes were detected in any of the samples besides the control sample. Therefore, the distillation processes of the present invention renders a cannabis plant product substantially free from odiferous compounds, and accordingly odorless and terpene-free as taught and described.

Other methods for analyzing the cannabinoid and terpene content of cannabis have been reported and would be understood by those of skill in the art, and could be substituted for the methods performed by Steep Hill Labs. See, e.g., Geise et al., Development and Validation of a Reliable and Robust Method for the Analysis of Cannabinoids and Terpenes in Cannabis, J. AOAC 98(6):1503 (2015).

As further confirmation, comparison of the samples from Examples 1-4 with the control sample was performed by human perceptive means, by smelling the initial plant material and resulting plant product directly, and further by smoking a portion of the samples to smell and taste the smoke. These comparisons, performed in a blinded manner, provided additional confirmation that the resulting plant product is indeed odor- and taste-free. It should be appreciated that if further or more formal confirmation is sought, olfactory testing could be performed by multiple blinded human subjects, whereby such subjects could be asked to rate and judge the odor of the cannabis and the odor and taste of the cannabis smoke.

By comparing the results of the cannabinoid analyses from Examples 1-4 in Tables 2, 4, 6, and 8 to the control sample result above, it is demonstrated that all experimental samples still contained cannabinoids, and the cannabinoid content of the resulting cannabis plant product is not significantly diminished compared to the initial cannabis plant material. Therefore, the distillation processes of the present invention does not significantly alter the cannabinoid content or profile of the plant material used. It should be understood that within the scope of the present invention are processes and apparatus that result in no detectable loss of cannabinoids, but also such processes and apparatus which may result in some loss but where such loss is considered insignificant or is otherwise within an acceptable tolerance or range, such as a loss of <1%, <5%, between 5-10%, between 10-25%, <25%, or the like.

Visual and tactile inspection of the samples of initial plant material and resulting plant product was also performed, to compare the quality and appearance of the cannabis buds. These tests revealed that the resulting plant product maintained its visual and tactile quality, without significant loss of variables such as color, texture, density, consistency, bud shape and size, and the like.

Additionally, a comparison of Examples 2 and 3 permitted the conclusion to be drawn that the final visual and tactile appeal of the cannabis plant product, while maintained in all Examples, was most noticably similar in quality and appearance to the starting plant material for steam distillation processes in particular.

Although different cannabis plant material will largely differ in qualities depending on strain, grow characteristics, and other factors, it should be understood that the comparison is made between the specific initial plant material used in the distillation process, and the resulting plant product of that process. It also should be appreciated that if further or more formal confirmation is sought, comparison could be performed by multiple blinded human subjects, whereby such subjects could be asked to rate and judge the various visual and tactile qualities of the cannabis plant material and plant product, based on variables such as color, texture, density, consistency, bud shape and size, and the like.

What is claimed is:

1. A process for producing an odorless plant product, comprising:
obtaining a predetermined amount of plant material to be rendered odorless;
placing the plant material within a distillation vessel;
introducing sufficient water into the distillation vessel to submerge the plant material;
heating the water in the distillation vessel to a predetermined temperature;
maintaining contact between the plant material and the heated water for a time sufficient to volatize the terpenes in the plant material; and
removing the plant material from the distillation vessel;
whereby the plant material has been rendered into an odorless plant product.

2. The process of claim 1, wherein the plant material is cannabis plant material.

3. The plant product of claim 1, wherein said plant product is an odorless cannabis plant product.

4. The plant product of claim 3, wherein said plant product is odorless as determined by the absence of detectable terpenes.

5. The plant product of claim 3, wherein said plant product is odorless as determined by a neutral untrained consumer.

6. The plant product of claim 3, wherein the smoke from the plant product, when said plant product is consumed by combustion, is odorless as determined by a neutral untrained consumer.

7. The plant product of claim 3, wherein said plant product has no significant loss of cannabinoids compared to the cannabis plant material.

8. The plant product of claim 3, wherein said plant product has no significant reduction in visual and tactile quality compared to the cannabis plant material.

9. A process for producing an odorless plant product, comprising:
obtaining a predetermined amount of plant material to be rendered odorless;
placing the plant material within a distillation vessel;
heating the distillation vessel to a predetermined temperature;
contacting the plant material with steam;
maintaining contact between the plant material and the steam for a time sufficient to volatize the terpenes in the plant material; and
removing the plant material from the distillation vessel;
whereby the plant material has been rendered into an odorless plant product.

10. The process of claim 9, wherein the plant material is cannabis plant material.

11. The plant product of claim 9, wherein said plant product is an odorless cannabis plant product.

12. The plant product of claim 11, wherein said plant product is odorless as determined by the absence of detectable terpenes.

13. The plant product of claim 11, wherein said plant product is odorless as determined by a neutral untrained consumer.

14. The plant product of claim 11, wherein the smoke from the plant product, when said plant product is consumed by combustion, is odorless as determined by a neutral untrained consumer.

15. The plant product of claim 11, wherein said plant product has no significant loss of cannabinoids compared to the cannabis plant material.

16. The plant product of claim 11, wherein said plant product has no significant reduction in visual and tactile quality compared to the cannabis plant material.

17. A cannabis plant product, having a cannabinoid profile and content comparable to other cannabis of the same strain, but being odorless as determined by a neutral untrained consumer.

18. The plant product of claim 17, wherein said plant product is further characterized as having no detectable terpenes.

19. The plant product of claim 17, wherein said plant product consists of whole, trimmed, cannabis buds.

20. The plant product of claim 19, wherein said plant product is further characterized as having no significant reduction in visual and tactile quality when compared to other whole, trimmed, buds of the same cannabis strain.
